(19) United States
(12) Patent Application Publication
(10) Pub. No.: US 2005/0222001 A1
(43) Pub. Date: Oct. 6, 2005

(54) Stable foamed compositions

(76) Inventors: Jan Baumeister, Farvagny-le-Grand (CH); Sarah Maillefer, Middes (CH); Andre Rehmann, Schmitten (CH)

Correspondence Address: STRIKER, STRIKER & STENBY, 103 EAST NECK ROAD, HUNTINGTON, NY, 11743, US

(21) Appl. No.: 11/098,171

(22) Filed: Apr. 4, 2005

(30) Foreign Application Priority Data
Apr. 5, 2004 (DE) 10 2004 016 683.8

(51) Int. Cl. A61K 7/075

(52) U.S. Cl. 510/123

(57) ABSTRACT

Stable foamed compositions are described. These compositions contain water and at least one alkoxylated silicone compound, selected from one or more of the following compound classes: bis-alkoxylated silicone compounds, alkoxylated silicone waxes, water-insoluble alkoxylated silicone compounds and esters of fatty acids and alkoxylated silicone compounds. The solid foamed compositions preferably contain additional consistency-imparting agents, which are waxy substances that are solid at 25° C. and/or thickeners. The mixture of ingredients, from which the stable foamed composition is made, is foamed with air and/or an inert gas and has a stable density of less than or equal to 0.8 g/cm².

### BACKGROUND OF THE INVENTION

The subject matter of the present invention is a permanent or stable aqueous composition, which is foamed with an inert gas, with a density of less than or equal to 0.8 g/cm³, and which comprises selected alkoxylated silicone compounds and preferably additional consistency imparting ingredients.

Cosmetic products have special requirements regarding sensory properties that are detected or sensed by users. Skin and hair care products, such as skin creams or hair care compositions, are frequently creamy emulsions or dispersions and each comprise an aqueous, hydrophilic phase and a hydrophobic phase containing oily or waxy substances. The sensory properties of these commercially available emulsion-form or dispersion-form cosmetic care products, especially their surface feel or touch properties, are disadvantageous. The products frequently feel too heavy, fatty and oily. Many creams have a disadvantageous colored or yellowish appearance because of the ingredients required for their action. Improved distributability on the hair and an improved emulsification are desirable for hair care compositions and shampoos that are worked into the hair. Hairstyling compositions are frequently in the form of gel products containing thickeners and have a disadvantageous sticky feel. The lively hairstyling market greatly demands products with new properties. Product innovations, which stimulate several senses simultaneously and awaken positive association, have great attention-getting potential.

Cosmetic hair treatment products in the form of unstable foams, which are temporarily generated by the user immediately prior to use, are known. These products are aerosol products, which are foamed by means of a propellant gas to form unstable foam when they are dispensed from a pressured container. Alternatively unstable foam can be produced by dispensing it from a container by means of a mechanical pump in connection with a foam head. This sort of temporary foam, which breaks down within a few minutes, has the disadvantage that the foam must be prepared prior to each application, which is troublesome for the user. Besides the required containers are expensive and susceptible to faults, since there is a danger of clogging the product delivery system. Also the recipe formulation freedom is limited, i.e. not all desired skin and hair care effects can be realized with temporary foam products.

Different starting points for making permanent or stable foamed cosmetic preparations are known, e.g. from WO 02/41847, WO 02/67882, EP 1 046 387, EP 1 216 682, CH 674 804 or JP 56-79613. The making of foamed products, which are storage stable especially at higher temperatures, simultaneously with good application properties for application to skin or hair that are satisfactory in all respects is however still not possible.

### SUMMARY OF THE INVENTION

It is an object of the present invention to provide easily useable stable foamed compositions for cosmetic or dermatological skin and hair care with high storage stability and with improved sensory properties (e.g. touch, optical, acoustic properties), without impairing the primary or main skin or hair care properties unacceptably, and without requiring an expensive, fault-susceptible packaging.

This object is attained by the use of certain silicone compounds as foam stabilizers for the stable or permanent foamed composition.

According to the present invention the stable or permanent foamed composition contains water and at least one alkoxylated silicone compound, which comprises one or more compounds from the following compound classes:
bis-alkoxylated silicone compounds,
alkoxylated silicone waxes,
esters of fatty acids and alkoxylated silicone compounds, and
water-insoluble alkoxylated silicone compounds,
   wherein the composition is foamed with air or an inert gas and has a stable density of less than or equal to 0.8 g/cm³.

Preferred compositions according to the invention contain
(A) at least one of the above-described alkoxylated silicone compounds,
(B) at least one consistency-imparting substance, selected from waxy substances that are solid at 25° C. and thickeners; and
(C) water.

Compositions according to the invention may be cosmetic, pharmaceutical or dermatological skin or hair treatment compositions, which can be present as finished foamed products present in a suitable package and can be dispensed from it. The foaming degree of these products after storage for at least one week at room temperature (20° C.) can still amount to at least 10% or more. The consistency of the foamed composition according to the invention can be solid, semisolid or creamy.

The product according to the invention has multiple sensory action or “polysensuality”. The term “polysensuality” means the impression conveyed is similar to the impression conveyed by a mousse au chocolat. Hand, skin and ear sense the lightness of the foamed mass, the increased creaminess with easy distributability and the soft crackle of the stable foamed mass during dispensing and application. The reduced density permits a more exact metering and improved distributability in relation to commercial products, such as waxes, gels or creams.

Besides water additional water-soluble cosmetically compatible organic solvents can be contained in a hydrophilic phase in amounts of 1 to 30% by weight or 5 to 20 percent by weight. These types of solvents are, e.g., lower monohydric alcohols, such as ethanol or isopropanol or polyhydric C₂- to C₄-alcohols, such as ethylene glycol, diethylene glycol, butylenes glycol or glycerol.

Alkoxylated Silicone Compounds

The alkoxylated silicone compounds preferably are contained in the compositions according to the invention in an amount of from 0.1 to 30% by weight, or from 0.2 to 20 percent by weight, especially preferably from 1 to 10% by weight, based on the total amount of unfoamed composition, i.e. the mixture of all ingredients of the stable foamed composition except for the air or inert gas introduced by foaming. Alkoxylated silicone compounds include polyalkoxylated silicone compounds, i.e. they have polyalkylene oxide groups. Silicone waxes are materials, which are waxy solid at room temperature, i.e. they have a melting or dripping point of greater than 25° C. Fatty acids are preferably carboxylic acids, dicarboxylic acids or hydroxycarboxylic acids with 8 to 32 carbon atoms. Water-insoluble compounds are substances, which dissolve only in amounts less than 1% by weight in pure water at 25° C.

Suitable bis-alkoxylated silicone compounds are poly-(dialkylsiloxanes), which have two terminal or side chains, which are polyoxyalkylene groups. Block copolymers, especially of type ABA, with a central block of polydimethylsiloxane and terminal blocks of polyethylene oxide and/or polypropylene oxide, are preferred. The terminal blocks can be unsubstituted, i.e. have end hydroxyl groups end or they can be substituted, e.g. with ether, ester or urethane groups, especially fatty acid esters. The alkoxylation degree is preferably from 2 to 40, especially of 10 to 30, especially preferably from 12 to 20.

Suitable silicone compounds are those of the general formula (I):
R1-(AO)ₓ₁—B1—SiMe₂O—(SiMe₂O)ₓ₃—SiMe₂O—B2-(AO)ₓ₂—R2 (1),
wherein R1 is a hydroxy group, an alkoxy group with 1 to 22 carbon atoms or a carboxy alkyl group with 2 to 22 carbon atoms, B1 and B2 are different or preferably the same and are a binding or a divalent connecting group, especially an alkylene terminal group with 1, 2, 3 or 4 carbon atoms; AO is an oxyalkylene group, especially oxyethylene or oxypropylene; R2 is hydrogen or an alkyl group with 1 to 22 carbon atoms esterified or etherified with the adjacent oxyalkylene group; x1 and x2 are numbers greater than or equal to 1 and their sum gives the alkoxylation degree and x3 is a number greater than or equal to 1 and gives the polymerization degree of the dimethylpolysiloxane.

Preferred silicone compounds are those with the INCI name bis-PEG-4 dimethicone, bis-PEG-12 dimethicone, bis-PEG-20 dimethicone, bis-PEG-12 dimethicone beeswax, bis-PEG-12 dimethicone candillilate, bis-PEG-15 dimethicone/IPDI copolymer, bis-PEG-15 methyl ether dimethicone, bis-PEG-18 methyl ether dimethylsilane, bis-PEG/PPG-14/14 dimethicone, bis-PEG/PPG-20/20 dimethicone, bis-PEG/PPG-16/16 PEG/PPG-16/16 dimethicone, bis-PPG-15 dimethicone/IPDI copolymer, bis-PPG-7 Undeceneth-21 dimethicone. Bis-(polyethylene oxide)-polydimethyl-siloxane, fatty acid esters of bis-(polyethylene oxide)-polydimethylsiloxane and mixtures of at least one bis-(polyethylene oxide)-polydimethylsiloxane and at least one fatty acid ester of bis-(polyethylene oxide)-polydimethyl siloxane, are preferred. Esterified bis-ethoxylated silicone waxes esterified with fatty acids are especially preferred.

Additional suitable silicone compounds include ethoxylated dimethylsilane methyl ether, e.g. water-dispersible silicone waxes of the general formula (II):
CH₃—(OCH₂CH₂)ₙ—O—Si(CH₃)₂—O—(CH₂CH₂O)ₘ—CH₃ (II),
Wherein n and m can be the same or different, give the ethoxylation degree and are preferably numbers between 5 and 40, especially from 10 to 20. An example is the compound with the INCl name bis-PEG-18 methyl ether dimethyl silane, which is marketed as Dow Corning® 2501 cosmetic wax.

Wax Materials

The waxy solid materials or substances of component (B) are preferably contained in the composition according to the invention in an amount of 0.05 to 50 percent by weight, or from 0.2 to 30 percent by weight, especially preferably from 0.5 to 10 percent by weight, based on the total amount of the unfoamed composition. Those materials are suitable as waxes, which have waxy properties, especially a solidification point greater than or equal to 25° C., preferably in a range above 30 to 100° C., especially in a range from 40 to 90° C.

The waxy substances, for example, include animal waxes, vegetable waxes, mineral waxes, synthetic waxes, micro-crystalline waxes, macro-crystalline waxes, paraffin waxes, ozocerite, montan waxes, Fischer-Tropsch waxes, polyolefin waxes (polyethylene, polybutylene waxes and the like), amide waxes, silicone waxes, beeswax, wool wax (lanolin) and its derivatives, such as wool wax alcohols, candelilla wax, carnauba wax, Japan wax, fats, fatty acid esters, fatty acid glycerides, long chain carboxylic acids or long-chain (C₁₀- to C₂₂) alcohols, with a melting or solidification points above 25° C. Especially ricinus wax, which is a hardened castor oil (INCl name: hydrogenated castor oil) with a melting point above about 90° C., is particularly preferred.

The composition can also contain liquid hydrophobic materials in a hydrophobic phase at room temperature. These liquid hydrophobic materials can be oils or oily substances, such as naturally occurring naturally made oils (plant and animal fat oil), synthetic oils, silicone oils, mineral oils, etheric oils, water-insoluble branched or linear aliphatic hydrocarbon substances, linear or branched alcohols, especially liquid fatty alcohols and long-chain ethers or esters, each of which preferably contains at least 8 carbon atoms. Suitable hydrocarbon substances include, e.g., liquid paraffins, squalene or squalane. Esters of trihydric and multihydric alcohols, especially plant triglycerides, such as olive oil, almond oil, peanut oil, sunflower seed oil and synthetic triglycerides, such as e.g. C₈- to C₁₀-trifatty acid glyceride esters or also jojoba oil, are also suitable.

Suitable hydrophobic substances include mono- or diesters of formula R¹-COOR², R¹—COO—R³—OOCR¹ and R²OOC—R³—COOR², wherein R¹ stands for a C₈- to C₂₂-alkyl group, R² stands for a C₃- to C₂₂-alkyl group and R³ for a C₂- to C₁₆-alkylene group. Naturally occurring monoester mixtures or waxy ester mixtures, such as jojoba oil or sperm oil, and branched primary alcohols, which are named guerbeta alcohols, are also suitable.

Substances, which are used usually as turbidity agents in cosmetic substances, are also suitable as hydrophobic substances. These substances include those of formula (III):
R¹—COO—(CHR⁴CHR⁵O)ₙ—COR⁶ (III),
wherein R¹ stands for a C₈- to C₂₂-alkyl group, R⁴ and R⁵ for hydrogen or methyl and R⁶ for hydrogen or for R¹ and n denotes a number between 1 and 12, preferably 1, 2, 3 or 4. Glycol difatty acid esters and polyethylene glycol difatty acid esters, which are present in solid form at room temperature, are preferred.

Thickeners

Thickeners for component (B) are, when contained in the composition, preferably contained in an amount of 0.05 to 30 percent by weight, of 0.2 to 20 percent by weight or especially preferably from 0.5 to 10 percent by weight, based on a total amount of the unfoamed composition. Suitable thickeners are especially those, which impart a flow limit to the composition. Suitable thickeners include
synthetic polymers, such as polyvinyl pyrrolidone or cross-linked polyacrylates (Carbomers, Carbopols);
polymers on a natural basis, especially polysaccharides and their derivatives, e.g. sclerotium gum, starches, gelatins, cellulose and its derivatives, such as carboxymethylcellulose, hydroxypropylcellulose, methyl cellulose, hydroxypropylmethylcellulose or hydroxyethyl cellulose, microcrystalline cellulose and extracts from algae, such as agar-agar, carrageenan or alginates and carouba gum, guar gum and its derivatives, such as alkylated or hydroxyalkylated guar, crystal gum, xanthan gum, gum arabic, pectin;
inorganic thickeners, such as hectorite, bentonite, aluminum and magnesium silicates; or
a mixture of the above-named substances.

Preferred thickeners include cross-linked polyacrylic acids or their salts, polysaccharides, polysaccharide derivatives and agar-agar.

Hair treatment compositions according to the invention containing from 0.5 to 10 percent by weight of carrageenan, especially kappa-carrageenan and/or iota-carrageenan, are especially preferred.

Hair-Fixing and Hair-care Polymers

In some embodiments the composition according to the invention contains at least one hair-fixing polymer and/or at least one hair-care polymer. The hair-fixing polymer or hair-care polymer is preferably contained in an amount of from 0.1 to 20 or 0.05 to 10 percent by weight, especially 0.1 to 5 percent by weight. The hair-fixing polymers can be anionic polymers, i.e. polymers with anionic or anionizable groups; cationic polymers, i.e. polymers with cationic or cationizable groups; zwitterionic polymers, i.e. polymers with cationic groups and anionic groups; amphoteric polymers, i.e. polymers with acid groups and basic groups or nonionic polymers. The anionizable groups are acid groups, such as carboxylic acid groups, sulfuric acid groups or phosphoric acid groups, which can be deprotonated by common bases, such as organic amines or alkali or alkaline earth hydroxides.

The anionic polymers can be partially or completely neutralized with a basic neutralization agent. Preferably they are present in 50 to 100% neutralized form, especially preferably 70 to 100%. Organic or inorganic bases suitable for cosmetic purposes can be used as neutralization agents. For example, suitable bases include amino alcohols, such as aminomethylpropanol (AMP), triethanolamine or monoethanolamine. However ammonia, NaOH and other bases are also suitable.

The anionic polymers can be homopolymers or copolymers with monomer units containing acid groups on a synthetic or natural basis, which are copolymerized as needed with comonomers, which contain no acid groups. A sulfuric acid group, a phosphoric acid group and a carboxylic acid group can be used as the acid group. The carboxylic acid group is particularly preferred. Suitable monomers containing acid groups include e.g. acrylic acid, methacrylic acid, crotonic acid, maleic acid, maleic acid anhydride, maleic acid monoesters, especially the mono-C₁ to C₇-alkyl esters of maleic acid, aldehydocarboxylic acids or ketocarboxylic acids. The comonomers not substituted with acid groups include, e.g., acryl amide, methacrylamide, alkyl- and dialkylacrylamides, alkyl- and dialkylmethacrylamides, alkylacrylates, alkylmethacrylates, vinyl caprolactone, vinyl pyrrolidone, vinyl ester, vinyl alcohol, propylene glycol or ethylene glycol, amine-substituted vinyl monomers, such as dialkylamino-alkylacrylates, dialkylaminoalkyl-methacrylates, monoalkylaminoalkylacrylates and monoalkylaminoalkylmethacrylates, in which the alkyl groups of these monomers are preferably C₁- to C₇-alkyl groups, especially preferably C₁- to C₃-alkyl groups.

Suitable polymers with acid groups are especially homopolymers of acrylic acid or methacrylic acid, which are not cross-linked or cross-linked with polyfunctional agents, copolymers of acrylic acid or methacrylic acid with monomers selected from the group consisting of acrylic acid or methacrylic acid esters, acrylamide, methacrylamide and vinyl pyrrolidones, homopolymers of crotonic acid and copolymers of crotonic acid with monomers selected from the group consisting of vinyl esters, acrylic acid esters, methacrylic acid esters, acrylamides and methacrylamides. A suitable natural polymer is, for example, shellac.

Preferred polymers with acid groups are: terpolymers of acrylic acid, alkyl acrylate and N-alkylacrylamide (INCl-name: acrylates/acrylamide copolymer), especially terpolymers of acrylic acid, ethyl acrylate and N-t-butyl-acrylamide; cross-linked or uncrosslinked vinyl acetate/crotonic acid copolymer (INCl name: VA/crotonates copolymer); copolymers of one or more C₁- to C₅-alkylacrylates, especially C₂- to C₄-alkylacrylates and at least one monomer selected from acrylic acid or methacrylic acid (INCl name: acrylates copolymer), e.g. terpolymers from tert.-butylacrylate, ethyl acrylate and methacrylic acid; sodium polystyrene sulfonate; vinyl acetate/crotonic acid/vinyl alkanoate copolymers, e.g. copolymers of vinyl acetate, crotonic acid and vinyl propionate; copolymers of vinyl acetate, crotonic acid and vinyl neodecanoate (INCl names: VA/crotonates/vinyl propionate copolymer, VA/crotonates/vinyl neodecanoate copolymer); aminomethyl propanol-acrylate copolymers; copolymers of vinyl pyrrolidone and at least one other monomer selected from the group consisting of acrylic acid, methacrylic acid, and, as needed, acrylic acid esters and methacrylic acid esters; copolymers of methyl vinyl ether and maleic acid monoalkyl esters (INCl name: ethyl ester of PVM/MA copolymer, butyl ester of PVM/MA copolymer); aminomethylpropanol salts of copolymers of allyl methacrylate and at least one other monomer selected from the group consisting of acrylic acid, methacrylic acid, and, as needed, acrylic acid esters and methacrylic acid esters; cross-linked copolymers of ethyl acrylate and methacrylic acid; copolymers of vinyl acetate, mono-n-butylmaleate and isobornyl acrylate; copolymers of two or more monomers selected from the group consisting of acrylic acid, methacrylic acid, and, as needed, acrylic acid esters and methacrylic acid esters; copolymers of octyl acrylamide and at least one monomer selected from the group consisting of acrylic acid, methacrylic acid, and, as needed, acrylic acid esters and methacrylic acid esters; polyesters of diglycol, cyclohexanedimethanol, isophthalic acid and sulfoisophthalic acid, in which the alkyl groups of the above-named polymers preferably contain 1, 2, 3 or 4 carbon atoms.

Preferred zwitterionic or amphoteric polymers are: Copolymers formed from alkylacrylamides, alkylaminoalkylmethacrylate and two or more monomers selected from acrylic acid, methacrylic acid, and, as needed, acrylic acid esters and methacrylic acid esters, especially copolymers of octylacryl amide, acrylic acid, butylaminoethylmethacrylate, methylmethacrylate and hydroxypropylmethacrylate (INCl name: octylacrylamide/acrylates/butylaminoethyl methacrylate copolymer); copolymers, which are formed from at least one first type of monomer, which has quaternary amino groups and at least one second type of monomer, which has acid groups; copolymers of fatty alcohol acrylates, alkylaminoxide methacrylates and at least one monomer selected from acrylic acid, methacrylic acid, and, as needed, acrylic acid esters and methacrylic acid esters, especially copolymers of lauryl acrylate, stearyl acrylate, ethyl-amine oxide methacrylate and at least one monomer selected from acrylic acid, methacrylic acid and esters thereof as needed; copolymers of methacryloyl-ethylbetaine and at least one monomers selected from methacrylic acid and methacrylic acid esters; copolymers of acrylic acid, methacrylate and methacrylamido-propyltrimethylammonium chloride (INCl: polyquaternium-47); copolymers made from acrylamidopropyltrimethylammonium chloride and acrylates or copolymers made from acrylamide, acrylamido-propyltrimethylammonium chloride, 2-amidopropyl-acrylamide sulfonate and dimethylaminopropylamine (INCl: Polyquaternium-43); and oligomers or polymers made from quaternary crotonic betaines or their esters.

Cationic polymers especially are polymers with primary, secondary, tertiary or quaternary amine groups. The cationic charge density amounts preferably to 1 to 7 meq/g. Suitable cationic polymers preferably contain quaternary amine groups. The cationic polymers can be homopolymers or copolymers, which contain quaternary nitrogen groups in the polymer chain or preferably as substituents on one or more monomers. The monomers containing ammonium groups can be copolymerized with non-cationic monomers. Suitable cationic monomers are unsaturated, radical polymerizable compounds, which contain at least one cationic group, especially ammonium-substituted vinyl monomers include, e.g., trialkylmethacryloxyalkyl ammonium, trialkylacryloxyalkyl ammonium, dialkyldiallyl ammonium and quaternary vinyl ammonium monomers with cyclic, cationic nitrogen containing groups, such as pyridinium, imidazolium or quaternary pyrrolidone, e.g. alkylvinylimidazolium, alkylvinylpyridinium, or alkylvinylpyrrolidone salts. The alkyl groups are preferably lower alkyl groups, especially C₁- to C₇-alkyl groups, preferably C₁- to C₃-alkyl groups.

The monomers containing ammonium groups can be copolymerized with non-cationic monomers. Suitable comonomers are for example acryl amide, methacryl amide, alkyl- and dialkylacrylamide, alkyl- and dialkylmethacryl amide, alkyl acrylate, alkylmethacrylate, vinyl caprolactone, vinyl caprolactam, vinyl pyrrolidone, vinyl ester, e.g. vinyl acetate, vinyl alcohol, propylene glycol or ethylene glycol, wherein the alkyl groups of these monomers preferably have one to seven carbon atoms, especially one to three carbon atoms.

Suitable polymers with quaternary amine groups are, for example, polymers described in the CTFA Cosmetic Ingredient Dictionary under the name “polyquaternium”, such as methylvinylimidazolium chloride/vinyl pyrrolidone copolymer (Polyquaternium-16) or quaternized vinyl pyrrolidone/dimethylamino-ethylmthacrylate copolymer (Polyquaternium-11) and quaternary silicone polymers and/or oligomers, such as silicone polymers with quaternary terminal groups (Quaternium-80).

Preferred cationic polymers on a synthetic basis include: poly(dimethyldiallylammonium chloride); copolymers of acrylamide and dimethyldially ammonium chloride; quaternary ammonium polymers, formed by reaction of diethyl sulfate and a copolymer of vinyl pyrrolidone and dimethylaminoethylmethacrylate, especially vinyl pyrrolidone/dimethylamino-ethylmethacrylate methosulfate copolymer, (e.g. GAFQUAT® 755 N and GAFQUAT® 734); quaternary ammonium polymers of polyvinyl pyrrolidone and imidazoliminemethochloride (e.g. LUVIQUAT® HM 550); Polyquaternium-35; Polyquaternium-57; polymer from trimethyl ammonium-ethyl-methacrylate chloride; the terpolymer of dimethyldiallylammonium chloride, sodium acrylate and acrylamide (MERQUAT Plus 3300); terpolymer of vinyl pyrrolidone, dimethylaminopropylmethacrylamide and methacryloylaminopropyllauryldimethyl ammonium chloride; terpolymer of vinyl pyrrolidone, dimethylaminoethyl-methacrylate and vinyl caprolactam (e.g. GAFFIX® VC 713); vinyl pyrrolidone/methacrylamidopropyltrimethyl ammonium chloride copolymer (GAFQUAT® HS 100); copolymers of vinyl pyrrolidone and dimethylaminoethylmethacrylate; copolymers of vinyl pyrrolidone, vinyl caprolactam and dimethylaminopropyl acrylamide; polyesters or oligoesters built up from at least one first monomer type, which is a hydroxy acid substituted with at least one quaternary ammonium group; substituted dimethylpolysiloxanes with quaternary ammonium end groups.

Suitable cationic polymers, which are derived from natural polymers, are especially cationic derivatives of polysaccharides, e.g. cellulose, starch or guar. Chitosan and chitosan derivative compounds are suitable. Cationic poly-saccharides have the general formula (IV):
G-O—B—N⁺R^{a}R^{b}R^{c}X⁽⁻⁾ (IV),
wherein G is an anhydroglucose residue, for example starch or cellulose anhydroglucose; B is a divalent connecting group, for example, an alkylene, an oxyalkylene, a polyoxyalkylene or hydroxyalkylene;
R^{a}, R^{b} and R^{c} are each, independently of each other, alkyl, aryl, alkylaryl, arylalkyl, alkoxyalkyl or alkoxyaryl with up to 18 carbon atoms respectively, wherein the total number of carbon atoms in R^{a}, R^{b} and R^{c} is at most 20; X is a common counter anion, for example a halogen, acetate, phosphate, nirate or alkyl sulfate, preferably chloride. Preferred cationic celluloses are those marketed under the INCl name, polyquaternium-10 and polyquaternium-24. A suitable cationic guar derivative is e.g. the derivative with the INCl name, guar hydroxypropyltrimonium chloride.

Chitosan, chitosan salts and chitosan derivative compounds are especially preferred as cation-active materials. The chitosan used in the composition of the invention is partially or completely deacetylated. The molecular weights of chitosan can vary over a wide range, for example from 20,000 to 5,000,000 g/mol. A low molecular weight chitosan is, for example, considered to be a chitosan with a molecular weight of from 30,000 to 70,000 g/mol. Preferably the molecular weight of the chitosan is above 100,000 g/mol, especially preferably form 200,000 to 700,000 g/mol. The deacetylation degree amounts to from 10 to 99%, especially preferably form 60 to 99%. A preferred chitosan salt is chitosonium pyrrolidone carboxylate, which for example is marketed under the trade name KYTAMER® PC of Amerchol, USA. The chitosan obtained has a molecular weight of about 200,000 to 300,000 g/mol and is deacetylated up to 70 to 85%. Quaternary, alkylated or hydroxyalkylated derivative chitosan compounds, for example, the hydroxyethyl chitosan, hydroxypropyl chitosan or hydroxybutyl chitosan, are suitable in the compositions according to the invention. The chitosans or chitosan derivative compounds should be present in neutralized or partially neutralized form when used in the compositions of the invention. The neutralization degree for the chitosan or the chitosan derivative compounds is preferably at least 50%, especially preferably between 70 and 100%, relative to the number of free base groups. In principle, all cosmetically compatible inorganic or organic acids may be used as neutralization agent, for example formic acid, tartaric acid, malic acid, lactic acid, citric acid, pyrrolidone carboxylic acid, hydrochloric acid, among other. Pyrrolidone carboxylic acid is especially preferred as neutralization agent.

Preferred cationic polymers on a natural basis include: cationic cellulose derivatives comprising hydroxyethyl cellulose and diallyldimethyl ammonium chloride; cationic cellulose derivates comprising hydroxyethyl cellulose and with trimethyl ammonium substituted epoxide; chitosan and its salts; hydroxyalkyl chitosans and their salts; alkylhydroxyalkyl chitosans and their salts; N-hydroxyalkyl chitosan alkyl ether; N-hydroxyalkylchitosan benzyl ether.

Suitable synthetic nonionic polymers include homopolymers or copolymers, which are built up from at least one of the following monomers: vinyl pyrrolidone, vinyl caprolactam, vinyl esters, such as vinyl acetate, vinyl alcohol, acrylamide, methacrylamide, alkyl- and dialkylacrylamide, alkyl- and dialkylmethacrylamide, alkylacrylate, alkylmethacrylate, propylene glycol or ethylene glycol, wherein the alkyl groups in these monomers preferably have from one to seven carbon atoms, especially preferably from one to three carbon atoms. For example, homopolymers of vinyl caprolactam, of vinyl pyrrolidone or of N-vinylformamide, are especially suitable. Additional suitable synthetic film-forming nonionic hair-fixing polymers are, e.g., copolymerizates of vinyl pyrrolidone and vinyl acetate, terpolymers of vinyl pyrrolidone, vinyl acetate and vinyl propionate, polyacrylamide, polyvinyl alcohols, and polyethylene glycol/polypropylene glycol copolymers. Suitable natural film-forming polymers include e.g. cellulose derivatives, e.g. hydroxyalkyl cellulose.

Preferred nonionic polymers include: polyvinyl pyrrolidone, polyvinyl caprolactam, vinyl pyrrolidone/vinyl acetate copolymers, polyvinyl alcohol, isobutylene/ethylmaleimide/hydroxyethyl maleimide copolymer; copolymers of vinyl pyrrolidone, vinyl acetate and vinyl propionate.

The stable foamed product according to the invention which contains at least one hair-fixing polymer is characterized by special haptic or touch properties and improved application properties as well as an outstandingly strong to extra strong hair-fixing action.

Foaming with Gases

The composition according to the invention is foamed with air or an inert gas until the foam degree of at least 10% and up to 500%, preferably between 20 and 200%, especially preferably between 30 and 100%, is reached and stable foam is formed. The foam degree in the sense of the present invention means the volume ratio and is calculated from the density of the composition before and after foaming as follows with the following formula (V):
[(D₀/D)−1]*100% (V),
wherein D₀ is the density prior to foaming and D is the density after foaming. Besides air inert gases such as nitrogen, carbon dioxide, nitrogen oxides, noble gases or mixtures of these gases can be used for forming. It is especially advantageous to use inert oxygen-free gases, such as nitrogen or carbon dioxide, to make foamed products containing oxygen sensitive ingredients.

The term “stable foam” or “permanent foam” relates to the product mass, which is characterized in that a gaseous substance in the form of gas bubbles is uniformly distributed in it and remains homogeneously distributed over a time interval of at least one week, preferably at least one month, especially preferably at least six months during storage at room temperature (20° C.), i.e. the foam degree amounts to at least 10%, preferably at least 20%. Gas bubbles of a size of preferably between 0.0001 and 10 mm, especially preferably between 0.01 and 1 mm, are contained in the foam product mass. The average diameter of the gas bubbles amounts preferably to from 0.1 to 0.8 mm, especially preferably from 0.2 to 0.4 mm. The density of the composition according to the invention is adjusted to preferably less than or equal to 0.8 g/ml, especially to 0.2 to 0.7 g/ml, especially preferably to 0.2 to 0.4 g/ml, by introducing a gas in the base composition.

Surfactant

A special advantage of the invention is that it is possible to make a surfactant-free stable foamed product, i.e. without additional surface-active compounds contained in it besides the obligatory ingredients. One embodiment of the invention concerns compositions, which are substantially free of additional surface-active compounds, especially free of anionic surfactants. This leads to special skin compatible products. The products are substantially free of surface-active compounds, if they contain either no or less than 1% by weight surfactants.

Understandably surfactants can be added for special applications. For example, hair care compositions can contain cationic surfactants or body cleansing agents can contain wash-active anionic, amphoteric or nonionic surfactants or emulsifiers for emulsifying or solubilizing effective ingredients or auxiliary ingredients. Suitable surfactants are, for example, those surfactants described in “International Cosmetic Ingredient Dictionary and Handbook”, 7^{th} Edition, Volume 2, in the section labeled “Surfactants”, especially under the subsection “Surfactants—Emulsifying Agents”. The surfactants can be present in an amount of 0.1 to 30 percent by weight, preferably from 0.2 to 5 percent by weight, in relation to the unfoamed composition. Higher sufactant amounts, such as e.g. 5 to 25 percent by weight, are contained, when the composition of the invention is used as a skin and hair cleansing composition (shampoo).

Nonionic surfactants include e.g. ethoxylated faty alcohols, ethoxylated nonyl phenols, alkylpolyglycosides, fatty acid monoglycerides and fatty acid diglycerides, ethoxylated and hydrogenated or non-hydrogenated castor oil, fatty acid alkanol amides, ethoxylated fatty acid esters. Cationic surfactants include e.g. long-chain quaternary ammonium compounds, such as those under the CTFA designation “Quaternium”, like alkyltrimethylammonium salts or dialkyldimethyl ammonium salts with C₈- to C₂₂-alkyl groups. Suitable cationic surfactants can be represented by the following general formula (VI):
N⁺R¹R²R³, R⁴X⁽⁻⁾ (VI)
wherein each of R¹ to R⁴, independently of each other, denote an aliphatic group, an aromatic group, an alkoxy group, a polyoxyalkylene group, an alkylamido group, a hydroxyalkyl group, an aryl group or an alkaryl group, each with from 1 to 22 carbon atoms, wherein each of the R¹ to R⁴ groups has at least eight carbon atoms. X⁽⁻⁾ is a cosmetically compatible anion. The anion can be a halogen, acetate, phosphate, nitrate or alkyl sulfate anion. However chloride is preferred. The aliphatic groups can also contain cross-linkages or other groups, for example amino groups, besides the carbon atoms and the hydrogen atoms. For example, suitable cationic surfactants include the chlorides or bromides of alkyldimethylbenzylammonium salts, alkyltrimethylammonium salts, e.g. cetyltrimethylammonium chloride or bromide, tetradecyltrimethylammonium chloride or bromide, alkyldimethylhydroxyethylammonium chloride or bromide, dialkyldimethylammonium chloride or bromide, alkylpyridinium salts, especially lauryl or cetyl pyridinium chloride, alkylamidoethyltrimethylammonium ether sulfate and cationic compounds, such as amine oxides, especially alkylmethylamine oxides or alkylaminoethyldimethylamine oxides. Cetyltrimethylammonium chloride is especially preferred. The so-called ester quats, for example C₈- to C₁₈-alkyl esters of betaine, for example palmityl betaine chloride, are especially preferred as cationic surfactants.

Suitable anionic surfactants, especially for hair and body cleansing agents, are alkali or alkaline earth salts of C₁₀- to C₁₈-alkyl sulfates, C₁₀- to C₁₈-alkylsulfonates, C₁₀- to C₁₈-alkylbenzenesulfonates, C₁₀-to C₁₈-xylenesulfonates and C₁₀- to C₁₈-alkyl ether sulfates ethoxylated with 1 to 10 ethylene oxide units, the ethoxylated sulfosuccinic acid semi-esters of the formula (VII):
R—(OCH₂CH₂)ₘ—O₂C—CH₂—CH(SO₃M)—COOM (VII),
wherein R represents a C₁₀- to C₁₈-alkyl group, M an alkali metal or alkaline earth method cation and m represents a number from 1 to 10 and the alkyl ether carboxylates of the general formula (VIII),
R—(OCH₂CH₂)ₘ—OCH₂—COOM (VIII),
wherein R represents a C₁₀- to C₁₈-alkyl group, M an alkali metal or alkaline earth metal cation and n represents a number from 1 to 10. The alkali and alkaline earth metal salts of the C₁₀- to C₁₈-alkyl ether sulfates ethoxylated with 1 to 10 ethylene oxide units are especially preferred.

Suitable amphoteric surfactants, especially for hair and body cleansing agents, are derivatives of aliphatic quaternary ammonium-, phosphonium- and sulfonium compounds of the generally formula (IX):
R¹—Y⁽⁺⁾(R²)ₓ—CH₂—R³-Z⁽⁻⁾ (IX),
wherein R¹ denotes a linear or branched alkyl-, alkenyl- or hydroxyalkyl group with 8 to 18 carbon atoms and 0 to 10 ethylene oxide units and 0 to 1 glyceride units; Y denotes an N—, P—, or S-containing group; R² is an alkyl or monohydroxyalkyl group with 1 to 3 carbon atoms; x is 1, in case Y is an S atom and x is 2 when Y is an N or a P atom; R³ denotes an alkyl or hydroxyalkyl group with 1 to 4 carbon atoms and Z denotes a carboxylate, sulfate, phosphonate or phosphate group. Additional suitable amphoteric surfactants are those, which are derived from betaine, e.g. C₈- to C₁₈-alkylbetaines, such as cocodimethylcarboxy-methylbetaine, lauryldimethylcarboxymethylbetaine, lauryldimethyl-alpha-carboxyethylbetaine, cetyldimethylcarboxymethylbetaine, oleyldimethyl-gamma-carboxypropylbetaine or lauryl-bis-(2-hydroxypropyl)-alpha-carboxyethylbetaine; C₈- to C₁₈-alkylsulfobetaines, such as cocodimethylsulfopropylbetaine, stearyl-dimethyl-sulfopropylbetaine, lauryidimethylsulfoethylbetaine, lauryl-bis-(2-hydroxyethyl)-sulfopropylbetaine; the carboxyl derivatives of imidazole, the C₈- to C₁₈-alkyldimethylammonium acetates, the C₈- to C₁₈-alkyldimethyl-carbonylmethylammonium salts and the C₈- to C₁₈-fatty acid alkylamidobetaines, such as coconut fatty acid amidopropylbetaine (INCl name: cocamido-propylbetaine) and the N-coconut fatty acid amidoethyl-N-[2-carboxymethoxy)-ethyl]glycinate (INCl name cocoamphocarboxyglycinate).

The subject matter of the invention also includes cosmetic, pharmaceutical or dermatological agents, which contain or comprise a stable foamed composition according to the invention. These cosmetic, pharmaceutical or dermatological agents also comprise at least one active ingredient, which is selected from the group consisting of pharmaceutically effective ingredients, dermatologically effective ingredients, skin care effective ingredients, hair care effective ingredients, permanent wave effective ingredients, hair dye compounds and hair dye pre-cursors.

Preferred embodiments of these agents contain
(A) at least one of the above-described alkoxylated silicone compounds;
(B) at least one consistency-imparting substance, as needed, which is at least one of waxy materials that are solid at 25° C. and thickeners;
(C) water, and
(D) at least one active substance selected from the group consisting of pharmaceutically effective ingredients, dermatologically effective ingredients, skin care effective ingredients, hair care effective ingredients, permanent wave effective ingredients, hair dye compounds and hair dye pre-cursors, so that when the agent is formed with air or an inert gas it has a stable density of less than or equal to 0.8 g/cm².

The at least one active substance preferably is present in the foamed composition according to the invention in an amount of e.g. 0.01 to 10 percent by weight, especially preferably from 0.1 to 5 percent by weight, based on the unfoamed composition. The active ingredients can be selected from the group consisting of plant extracts, vegetable extracts, protein hydrolyzates, silk hydrolyzates, light-protective agents, anti-oxidants, radical-trapping agents, anti-flaking agents, luster-imparting agents, vitamins, panthenol, softeners, combability-improving agents, skin moisturizing agents, proteins, insect repellants, bactericides, anti-viral agents, anti-microbial agents, proteolytically effective substances, keratolytically acting substances, keratin-reducing substances, oxidizing agents, direct-dyeing dye compounds and oxidation dye pre-cursor compounds.

Suitable hair-care additives especially are plant extracts, vegetable extracts, protein hydrolyzates, silk hydrolyzates, light-protective agents, anti-oxidants, radical trapping agents, anti-flaking agents, luster-imparting agents, vitamins, panthenol, softeners and combability improving ingredients. Cationic or cation-active hair care substances come into consideration as combability-improving ingredients. These cationic or cation-active hair care substances include cationic polymers, cationic surfactants, cationic silicone compounds cationic derivatives of proteins or protein hydrolyzates and betaine each with at least one cationic or cation-active groups. A hair and body cleansing agent in the form of a stable foamed product containing the composition according to the invention and at least one wash-active surfactant is an especially preferred embodiment of the invention.

Suitable active hair care or dermatological substances are softening, moisturizing or moisture-containing substances, anti-inflammatory substances, light protective agents, vitamins, proteins, insect repellants, bactericides, anti-viral ingredients, anti-microbial ingredients, proteolytically active substances, keratolytically active substance and medicines.

Hair dye compositions according to the invention can be in the form of an oxidative dye composition based on oxidative dyestuffs and also a non-oxidative dye composition based on direct-dyeing dyestuffs. The total amount of the oxidation dye precursors contained in an oxidative dye composition according to the invention amounts to from about 0.01 to 12 percent by weight, especially about 0.2 to 6 percent by weight. Suitable oxidation dye precursor compounds can for example by the developer substances and coupler substances disclosed in WO 02/67882. The total amount of direct-dyeing dyestuffs included in a dye composition according to the invention amounts to about 0.01 to 7 percent by weight, preferably about 0.2 to 4 percent by weight. The direct-dyeing dye compounds disclosed in WO 02/67882 are especially suitable in the hair dye compositions according to the invention.

A hair care composition, which contains at least one hair care effective ingredient, is a special embodiment of the composition according to the invention. Hair care compositions are e.g. conditioners, treatments, hair care agents, rinses or the like. The at least one hair care effective or active ingredient is at least one cationic surfactant, at least one amine-substituted surfactant, at least one cationic silicone compound, at least one amine-substituted silicone compound, at least one cationic polymer and/or at least one amine-substituted polymers. The at least one hair care effective ingredient can be contained in the composition in an amount between 0.01 to 10.0 percent by weight, especially between 0.01 to 5.0 percent by weight, based on a total amount of the finished product. The hair care composition according to the invention can remain on the dry, moist or wet hair after application or after a suitable acting time it can be rinsed from the hair. The acting time depends on the type of hair. As a general guideline the acting time can be between 0.5 and 30 minutes, especially between 0.5 and 10 minutes, preferably between 1 and 5 minutes. Especially a combination of an amidoamine and/or a quaternarized amidoamine according to one of the general formulae listed below and a terminally functionalized diquaternary silicone polymer is suitable in an outstanding manner to prepare a cosmetic hair care composition with outstandingly improved hair care action.

Cationic and/or amine-substituted polymers suitable as hair care active ingredients are, among others, the above-described polymers. Besides the above-described cationic surfactants additional suitable cationic or amine-substituted surfactants are those of the formula R1—NH—(CH₂)ₙ—NR2R3 or of the formula R1—NH—(CH₂)ₙ—N⁺R2R3R4 X⁻, wherein R1 denotes an acyl or alkyl group with 8 to 24 carbon atoms, which is branched or unbranched, saturated or unsaturated, wherein the acyl and/or the alkyl group can contain one or more OH groups; wherein R2, R3 and R4, independently of each other, are hydrogen, alkyl or alkoxyalkyl groups with 1 to 6 carbon atoms, which are the same or different saturated or unsaturated and can be substituted with one or more hydroxyl groups; X⁻ is an anion, especially a halide ion, and a compound of the general formula RSO₃⁻, wherein R denotes a saturated or unsaturated aliphatic group with 1 to 4 carbon atoms, and n denotes a whole number between 1 and 10, preferably from 2 to 5. Preferably the hair care ingredient is an amidoamine and/or a quaternary amidoamine of the above-described formula, wherein R1 is a branched or unbranched saturated or unsaturated acyl group with 8 to 24 carbon atoms, which can contain at least one OH group. Those amines and/or quaternarized amines, in which at least one of the groups R2, R3 and R4 denote a group of the formula CH₂CH₂OR5, wherein R5 is an alkyl group with 1 to 4 carbon atoms, are preferred.

Suitable amines or amidoamines, which can be quaternized, are e.g. those with the INCl name ricinolamidopropyl betaine, ricinolamidopropyl dimethylamine, ricinolamidopropyl dimethyl lactate, ricinolamidopropyl ethyldimonium ethosulfate, ricinolamidopropyltrimonium chloride, ricinolamidopropyltrimonium methosulfate, cocamidopropyl betaine, cocamidopropyl dimethylamine, cocamidopropyl ethyldimonium ethosulfate, cocamidopropyltrimonium chloride, behenamidopropyl dimethylamine, isostearylamidopropyl dimethylamine, stearylamidopropyl dimethylamine, Quaternium-33, undecyleneamidopropyltrimonium methosulfate.

Suitable cation-active silicone compounds have preferably at least one primary, secondary or tertiary amino group (amine-substituted silicone compound) or at least one quaternary ammonium group (cationic silicone compound). Suitable silicone polymers with amino groups are known under the INCl name amodimethicone or PEG-x amodimethicone, wherein x is the ethoxylation degree, which preferably is between 2 and 10. Here it is a matter of a polydimethylsiloxane with aminoalkyl side or end groups. Suitable aminosilicones are those of the general formula (X):
R⁸R⁹R¹⁰Si—(OSi R¹¹R¹²)ₓ—(OSiR¹³Q)_{y}—OSiR¹⁴R¹⁵R¹⁶ (X),
wherein R⁸, R⁹, R¹⁴ and R¹⁵, independently of each other, are equal or different and each represent C₁- to C₁₀-alkyl, phenyl, hydroxy, hydrogen, C₁- to C₁₀-alkoxy or acetoxy, preferably C₁- to C₄-alkyl, especially preferably methyl;
R¹⁰ and R¹⁶ are the same or different and, independently of each other, represent —(CH₂)ₐ—NH₂ with a=1 to 6, C₁- to C₁₀-alkyl, phenyl, hydroxy, hydrogen, C₁- to C₁₀-alkoxy or acetoxy, preferably C₁- to C₄-alkyl, especially preferably methyl; R¹¹, R¹² and R¹³ are the same or different and independently of each other each represent hydrogen, a C₁- to C₂₀-substituted hydrocarbon group with at least one O and/or N atom substituent and an C₁- to C₂₀-unsubstituted hydrocarbon group, preferably a C₁- to C₁₀-alkyl or phenyl group, especially preferably a C₁- to C₄-alkyl group, most preferably methyl;
Q represents -A-N R¹⁷R¹, or -A-N⁺R¹⁷R¹⁸ R¹⁹, wherein A stands for a divalent C₁- to C₂₀-alkylene compound group, which can contain an O—, N— or OH substituent group, and R¹⁷, R¹⁸ and R¹⁹, independently of each other, are equal or different and represent hydrogen, a C₁- to C₂₂-substituted hydrocarbon group, preferably a C₁- to C₄-alkyl or phenyl group;
X represents a number between 1 and 10,000, preferably between 1 and 1000; and Y represents a number between 1 and 500, preferably between 1 and 50.

Preferably Q stand for —(CH₂)₃—NH₂, —(CH₂)₃NHCH₂CH₂NH₂, —(CH₂)₃OCH₂—CHOHCH₂NH₂ and —(CH₂)₃N(CH₂CH₂OH)₂, —(CH₂)₃—NH₃⁺ and —(CH₂)₃OCH₂CHOH—CH₂N⁺(CH₃)₂R²⁰, wherein R²⁰ is a C₁- to C₂₂-alkyl residue.

The molecular weight of the amino-silicones is between 500 and 100,000 g/mol. The amine content (meq/g) is preferably in a range of from 0.05 to 2.3, especially preferably from 0.1 to 0.5.

Suitable silicone polymers with two terminal quaternary ammonium groups are known under the INCl name Quaternium-80. The silicone polymers are dimethylsiloxanes with two terminal aminoalkyl groups. The quaternary aminosilicones that are suitable have the following general formula (XI):
R²¹R²²R²³N⁺-A-Si R⁸R⁹—(OSi R¹¹R¹²)ₙ—OSi R⁸R⁹-A-N⁺R²¹R²²R²³ 2X⁻ (XI),
wherein A denotes a divalent C₁- to C₂₀-alkylene connecting group, which can contain an O, N or OH group substituent, and is preferably —(CH₂)₃OCH₂CHOH—CH₂N⁺(CH₃)₂R²⁰, wherein R²⁰ is a C₁- to C₂₂-alkyl residue, which can have an OH group substitutent;
wherein R⁸, R⁹, R¹¹ and R¹² have the same significance as above in formula (X) and are preferably methyl groups;
wherein R²¹, R²² and R²³, independently of each other, each represent a C₁- to C₂₂-alkyl residue, which can also contain hydroxy group substituents and wherein preferably at least one of the groups has at least 10 carbon atoms and the remaining groups have one to four carbon atoms; and
n is a number from 0 to 200, preferably 10 to 100.

These diquaternary polydimethylsiloxanes are marketed under the trademark ABIL® QUAT 3270, 3272 and 3274 of Goldschmidt, Germany.

Manufacture

The composition according to the invention and agent can be made by a method comprising the steps of (a) providing an unfoamed composition or mixture comprising water, at least one alkoxylated silicone compound and, as needed, at least one consistency-imparting ingredient, which is at least one waxy substance that is solid at 25° C. and/or at least one thickener; (b) subsequently or simultaneously heating the unfoamed composition to a temperature above a melting point of any waxy substance present and (c) subsequently or simultaneously foaming the unfoamed substance or mixture with air and/or at least one inert gas or forcing air and/or at least one inert gas into the unfoamed substance or mixture.

The making of the unfoamed composition or mixture in the form of the dispersion can occur according to known methods. A review of modern methods for making semisolid and liquid emulsions is available in the article in the SOFW Journal, 124 year, 5/98, pp. 308 to 313, and the article in the SOFW Journal, 118 year, 5/92, pp. 287 to 296. The manufacture occurs in the usual manner so that the hydrophobic fat phase is heated to about 75° C. and is combined with the hydrophilic aqueous phase similarly heated to about 75° C. under intensive mixing with a mixer or homogenizer. Subsequently cooling occurs to form a finished dispersion. The gas introduction occurs preferably at the time point at which the mixture is still not completely cooled, e.g. at 30 to 40° C.

The forcing in or foaming with air and/or inert gas can occur by means of a suitable apparatus for that purpose, e.g. by means of a rapidly running stirring device or stirrer, so that gas from the surrounding atmosphere (preferably air) is introduced into the mass. A first heated liquid mixture solidifies on cooling. The foaming can also occur when the liquid unfoamed composition is conducted through a mixer, which has a mixing head and a feed device with respective connectors for supplying it and the gas simultaneously to the mixing head. The composition can be acted on with a gas, preferably air, CO₂ or nitrogen, in a gas mixing unit (e.g. as Euromix or a dynamic foam generator top mix from a Hansa Industry mixer or with an Ultra Turax laboratory mixer). The use of nitrogen with oxygen sensitive ingredients is especially preferred. The forcing in of air and/or inert gas is preferably in a range between 20 and 200%. The forcing in or action of air and/or inert gas influences the structure and consistency and can be adjusted as desired. Preferred mixers comprise a rotor/stator mixing head and feed device. During the mixing process a gas is supplied by means of an additional connector to the rotor/stator mixing head. A Becomix Duohomogenizer, which has an additional connector, through which the gas can be directly input to the rotor and comes in contract with the phases present in the toothed wheel or rim of the homogenizer, is especially preferred. For this purpose the gas can be supplied by applying a low pressure (vacuum) at the stirring vessel and/or by means of an overpressure in the gas supply line. The foam density, consistency, viscosity and foam bubble size can be adjusted as desired by changing the flow rates, rotation speed, temperature and pressure.

The stable foamed products are filled in suitable packages or containers, such as tubes or cups or pans. The containers preferably comprise a transparent material, such as glass or a transparent plastic, in order to make the advantageous optical properties observable when the composition is in the container. The containers can also have an apparatus for dispensing the product, especially a mechanically driven pump apparatus. The permanent or stable foams are storage stable for longer time and feel creme-like or creamy to velvety according to their gas bubble size.

Consistency, Viscosity

The final product is a viscose composition, for which the desired final viscosity usually results after cooling. The desired final viscosity however can also be adjusted by addition of electrolytes selected from alkali metal or alkaline earth metal salts, such as sodium chloride or sodium sulfate, or other thickening substances, such as celluloses or cellulose derivatives.

For an especially good stability the foamed products both the unfoamed composition and the foamed composition should have a flow limit and a sufficiently high viscosity. The viscosity of the unfoamed composition preferably is in a range from 1000 to 30,000 mPa s, especially preferably in a range from 3000 to 20,000 mPa s. The viscosity of the foamed compositions preferably is between 1000 and 100,000 mPa s, especially preferably between 1500 and 80000 mPa s, as measured with a Haake viscometer VT 550, measurement system SVDIN at 25° C. and with a shear rate of 50 s⁻¹. The flow limit preferably is at least 10 Pa, especially preferably at least 50 Pa, measured with a Bohlin Rheometer CS, measurement system CP 4/40 at 25° C. with a linearly increasing shear stress of 0.1 to 600 Pa (200 s).

Advantageous Properties of the Stable Foamed Composition

The foamed products according to the invention are characterized by a content of very small uniformly distributed gas bubbles. The composition especially has the following advantageous sensory properties:
it has a very pleasing haptic impression or feel and feels creamy or soft, but not sticky, it has a low density, feels light and is easily distributed on keratinic surfaces, such as skin or hair;
it has a pleasant cosmetic white color of special purity; especially an improvement over colored or yellow tinted creams in an unfoamed state, since the cream may already white with a small amount of the finest air bubbles distributed in it;
acoustic effects are detectable, e.g. crackling during pressing the foamed composition from a tube containing it or distributing it on the skin or working it into the hair.

The agent also has the advantage that it can be applied to an inclined surface satisfactorily without running off or dripping.

### Preferred Embodiments

Preferred embodiments include:
a stable foamed cosmetic skin cream containing at least one skin care ingredient;
a stable foamed dermatological or medicinal salve containing at least one pharmaceutical or dermatological effective ingredient acting on skin or absorbed by the body through the skin when the salve is applied to the skin;
a stable foamed sunscreen cream containing at least one organic or inorganic UV filter;
a stable foamed hairstyling cream containing at least one hair-fixing substance;
a stable foamed hair care cream containing at least one hair care substance;
a stable foamed cream for permanent wave treatments, which contains at least one permanent wave effective ingredient;
a stable foamed cream for hair smoothing, which contains at least one hair smoothing effective ingredient;
a stable foamed hair coloring cream, containing at least one direct-dyeing hair dye or at least one oxidiation dye precursor.

The following examples illustrate the above-described invention in more detail, but the details in these examples should not be considered as limiting the claims appended hereinbelow.

### EXAMPLES

### Example 1

### Stable Foamed Hairstyling Product

| | |
|---|---|
| 3.5 g | Bis-PEG-12 Dimethicone Candelillate (Siliconyl |
| | Candelilla) |
| 2.0 g | Bis-PEG-20 Dimethicone (SF 1388, GE Silicones) |
| 1.5 g | Lanolin alcohol (DUSORAN ®) |
| 1.5 g | Vinyl acetate/crotonic acid/vinyl neodecanoate copolymer |
| | (RESYN ® 28-2930, National Starch) |
| 1 g | Kappa-Carrageenan (GENUGEL ® X-901-02) |
| 0.5 g | Beeswax |
| 0.25 g | Aminomethylpropanol |
| 0.001 g | Acid Red 52 (Cl 45100) |
| 10 g | Ethanol |
| 40 g | Water |

The ingredients are combined and heated at about 90° C. The heated mixture is then rapidly stirred with a stirring device and solidifies on cooling. To speed up the process the mixture can be actively cooled. One obtains a solidified foam (density about 0.2 to 0.3 g/ml), which has the consistency of a solid Mousse au chocolat. It is soft and creamy and crackles when rubbed in the hands. When the foam dries, about 90% of the volume of the mass is retained and a foamy-gummy non-sticky solid body is obtained. In a closed glass the foam keeps its volume and its properties over four weeks storage at 40° C. Only a slight shrinkage of the mass of about 5 to 10%, which is finished in a few days, is observed. The distributability of the moist mass is very easy, without foaming up or cracking. A creamy, colorless, transparent film is obtained on the hand. When applied to hair, the hair is strongly to extra strongly fixed. The foam is very suitable for curl definition and volume increasing.

### Example 2

### Stable Foamed Hairstyling Product

| | |
|---|---|
| 3.5 g | Bis-PEG-12 Dimethicone Candelillate (Siliconyl |
| | Candelilla, Koster Keunen) |
| 0.5 g | Bis-PEG-12 Dimethicone Beeswax (Siliconyl Beeswax, |
| | Koster Keunen) |
| 2.0 g | Bis-PEG-20 Dimethicone (SF 1388, GE Silicones) |
| 1.5 g | Lanolin alcohol (DUSORAN ®) |
| 2 g | Cetearyl alcohol (LANETTE ® O) |
| 1 g | Stearamidopropyl Dimethylamine (TEGOAMID ® S18)) |
| 1 g | Kappa-Carrageenan (GENUGEL ® X-901-02) |
| 1 g | PEG-7 amodimethicone (ULTRASIL ® A-21, Noveson) |
| 0.25 g | Aminomethylpropanol |
| 0.001 g | Acid Red 52 (Cl 45100) |
| 10 g | Ethanol |
| 40 g | Water |

### Example 3

### Stable Foamed Hair Smoothing Product

| | |
|---|---|
| 3.5 g | Bis-PEG-12 Dimethicone Candelillate (Siliconyl |
| | Candelilla) |
| 2.0 g | Bis-PEG-20 Dimethicone (SF 1388, GE Silicones) |
| 1.5 g | Lanolin alcohol (DUSORAN ®) |
| 1 g | lota-Carrageenan (GENUVISCO ® X-904-02) |
| 0.5 g | Beeswax |
| 2.5 g | Ammonia, 25% |
| 10 g | Ammonium thioglycolate, 70% (ATG) |
| 4 g | Diammonium dithioglycolate, 40% (DADTG) |
| 0.001 g | Acid Red 52 (Cl 45100) |
| 10 g | Ethanol |
| 40 g | Water |

The carrageenan is dissolved in hot water with addition of the Bis-PEG-20 and slowly mixed with ATG and DADTG. The melted waxes are stirred in and the remaining components added. The warm mixture is rapidly stirred with a stirring device and solidifies on cooling. This process can be accelerated by actively cooling the mixture. One obtains a white elastic foam (density about 0.7 g/ml), which has a consistency that reminds one of Mousse au chocolate

### Example 4

### Stable Foamed Hair Dyeing Product

| | |
|---|---|
| 3.5 g | Bis-PEG-12 Dimethicone Candelillate (Siliconyl |
| | Candelilla) |
| 2.0 g | Bis-PEG-20 Dimethicone (SF 1388, GE Silicones) |
| 1.5 g | Lanolin alcohol (DUSORAN ®) |
| 1 g | Kappa-Carrageenan (GENUGEL ® X-901-02) |
| 0.5 g | Beeswax |
| 0.25 g | Aminomethylpropanol |
| 0.3 g | Rubin red Y* |
| 0.15 g | 2-amino-6-chloro-4-nitrophenol (RODOL ® 9R Base) |
| 10 g | Ethanol |
| 40 g | Water |

*mixture of 70 wt. % HC Red No. 10 (3-[(4-amino-2-chloro-5-nitrophenyl)-amino]-1,2-propandiol) and 30 wt. % HC Red No. 11 (3,3′-[(2-chloro-5-nitro-1,4-phenylen)diimino]-bis-1,2-propandiol)

Omission of the alkoxylated silicone compounds used in the composition of the invention or their replacement by alkoxylated silicone compounds not according to the invention in examples 1 to 4 lead to foam products with insufficient stability.

However in the above examples the Bis-PEG-20 Dimethicone may be replaced by Bis-PEG-18 Methyl Ether Dimethyl Silane (Dow Corning® 2501 Cosmetic Wax).

The disclosure in German Patent Application 10 2004 016 683.8 of Apr. 5, 2004 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in stable foamed compositions, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

1. A stable foamed composition containing water, air and/or at least one inert gas, and at least one alkoxylated silicone compound, said at least one alkoxylated silicone compound comprising one or more members of the following compound classes:
bis-alkoxylated silicone compounds,
alkoxylated silicone waxes,
esters of fatty acids and alkoxylated silicone compounds, and
water-insoluble alkoxylated silicone compounds; and
wherein the stable foamed composition is provided with a density of less than or equal to 0.8 g/cm³ by foaming with said air and/or said at least one inert gas.

2. The stable foamed composition as defined in claim 1, wherein said at least one alkoxylated silicone compound is present in an amount of 0.1 to 30 percent by weight, based on a total amount of unfoamed composition, and wherein said at least one alkoxylated silicone compound is a block copolymer, a fatty acid ester of said block copolymer or an ethoxylated dimethylsilane methyl ether, said block copolymer comprising a central block of polydimethylsiloxane and terminal blocks of polyethylene oxide and/or polypropylene oxide.

3. The stable foamed composition as defined in claim 1, wherein said at least one alkoxylated silicone compound is selected from the group consisting of bis-(polyethyleneoxide)-polydimethylsiloxanes and fatty acid esters of bis-(polyethyleneoxide)-polydimethylsiloxanes.

4. The stable foamed composition as defined in claim 1, further comprising at least one consistency-imparting ingredient and wherein said consistency-imparting ingredient comprises at least one waxy substance that is solid at 25° C. and/or at least one thickener.

5. The stable foamed composition as defined in claim 4, wherein said at least one waxy substance, when present, is contained in an amount of 0.05 to 50 percent by weight, based on a total amount of unfoamed composition; said at least one waxy substance has a solidification point at or above 25° C. and said at least one waxy substance comprises animal wax, vegetable wax, mineral wax, synthetic wax, micro-crystalline wax, macro-crystalline wax, paraffin wax, ozocerite, montan wax, Fischer-Tropsch wax, polyolefin wax, amide wax, silicone wax, beeswax, wool wax, wool wax alcohol, candelilla wax, carnauba wax, Japan wax, fat, a fatty acid ester, a fatty acid glyceride, a C₁₀- to C₂₂-carboxylic acid with a solidification point at or above 25° C. and/or a C₁₀- to C₂₂-alcohol with a solidification point at or above 25° C.

6. The stable foamed composition as defined in claim 4, wherein said consistency-imparting ingredient comprises said at least one waxy substance that is solid at 25° C. and said at least one thickener.

7. The stable foamed composition as defined in claim 4, wherein said at least one thickener, when present, is contained in an amount of 0.05 to 30 percent by weight, based on a total amount of unfoamed composition, and said at least one thickener comprises at least one synthetic polymer, at least one polymer on a natural basis and/or at least one inorganic thickener;
wherein said at least one synthetic polymer is selected from the group consisting of polyvinyl pyrrolidone and cross-linked polyacrylates;
wherein said at least one polymer on said natural basis is selected from the group consisting of sclerotium gum, starches, gelatins, cellulose, carboxymethyl cellulose, hydroxypropyl cellulose, methyl cellulose, hydroxypropylmethyl cellulose, hydroxyethyl cellulose, microcrystalline cellulose, agar-agar, carrageenan, alginate, carouba gum, guar gum, alkylated guar, hydroxyalkylated guar, karaya gum, xanthan gum, gum arabicum and pectin; and
wherein said at least one inorganic thickener is selected from the group consisting of hectorite, bentonite, aluminum silicates and magnesium silicates.

8. The stable foamed composition as defined in claim 7, containing from 0.5 to 10 percent by weight of said carrageenan, based on said unfoamed composition.

9. The stable foamed composition as defined in claim 1, further comprising at least one hair-fixing and/or at least one hair-care polymer.

10. The stable foamed composition as defined in claim 9, containing from 0.01 to 20 percent by weight, based on said unfoamed composition, of said at least one hair-fixing and/or said at least one hair-care polymer, and wherein said at least one hair-fixing and/or said at least one hair-care polymer comprises at least one polymer with at least one anionic or anionizable group, at least one polymer with at least one cationic or cationizable group, at least one zwitterionic and/or amphoteric polymer and/or at least one nonionic polymer;
wherein said at least one polymer with at least one anionic or anionizable group is selected from terpolymers of acrylic acid, ethyl acrylate and N-tert.-butylacrylamide; cross-linked or uncross-linked vinyl acetate/crotonic acid copolymers, terpolymers of tert.-butyl acrylate, ethyl acrylate and methacrylic acid; sodium polystyrene sulfonate; copolymers of vinyl acetate, crotonic acid and vinyl propionate; copolymers of vinyl pyrrolidone and at least one of acrylic acid, methacrylic acid, acrylic acid esters and methacrylic acid esters; copolymers of methyl vinyl ether and maleic acid monoalkyl esters; aminomethylpropanol salts of copolymers of allyl methacrylate and at least one of acrylic acid, methacrylic acid, acrylic acid esters and methacrylic acid esters; cross-linked copolymers of ethyl acrylate and methacrylic acid; copolymers of vinyl acetate, mono-n-butyl maleate and isobornyl acrylate; copolymers of two or more of acrylic acid, methacrylic acid, acrylic acid esters and methacrylic acid esters; copolymers of octylacrylamide and at least one of acrylic acid, methacrylic acid, acrylic acid esters and methacrylic acid esters and polyesters of diglycol, cyclohexane dimethanol, isophthalic acid and sulfoisophthalic acid;
wherein said at least one polymer with at least one cationic or cationizable group is selected from the group consisting of cationic cellulose derivatives of hydroxyethyl cellulose and diallyldimethylammonium chloride; cationic cellulose derivatives of hydroxyethyl cellulose and trimethyl ammonium substituted epoxides; poly(dimethyldiallylammonium chloride); copolymers of acrylamides and dimethyldiallylammonium chloride; quaternary ammonium polymers formed by reaction of diethyl sulfate and a copolymer of vinyl pyrrolidone and dimethylaminoethylmethacrylate; quaternary ammonium polymers of methylvinylimidazolium chloride and vinyl pyrrolidone; polyquaternium-35; polymers of trimethylammoniumethylmethacrylate chloride; polyquaternium-57; substituted polydiemthylsiloxanes terminated with quaternary ammonium groups; copolymers of vinyl pyrrolidone, dimethylaminopropylmethacrylamide and methacryloylaminopropyllauryldimethylammonium chloride; chitosan; chitosan salts; N-hydroxyalkylch itosanalkyl ether; N-hydroxyalkylchitosan benzyl ether; copolymers of vinyl caprolactam, vinyl pyrrolidone and dimethylaminoethyl-methacrylate; copolymers of vinyl pyrrolidone, vinyl caprolactam and dimethylaminopropylacrylamide; polyesters built up from at least one hydroxy acid substituted with at least one quaternary ammonium group; and terpolymers of vinyl pyrrolidone, methacrylic amide and vinyl imidazole;
wherein said at least one zwitterionic and/or amphoteric polymer is selected from the group consisting of copolymers of octylacrylamide, acrylic acid, butylaminoethylmethacrylate, methylmethacrylate and hydroxypropyl-methacrylate; copolymers of lauryl acrylate, stearyl acrylate, ethylaminoxide methacrylate and at least one of acrylic acid, methacrylic acid, acrylic acid esters and methacrylic acid esters; copolymers of methacryloylethylbetaine and at least one of methacrylic acid and methacrylic acid esters; copolymers of acrylic acid, methacrylates and methacrylamidopropyltrimethylammonium chloride; polymers made from quaternary crotonic betaines; and polymers made from quaternary crotonic betaine esters; and
wherein said at least one nonionic polymer is selected from the group consisting of polyvinyl pyrrolidone, polyvinyl caprolactam, vinyl pyrrolidone/vinyl acetate copolymers, polyvinyl alcohol, isobutylene/ethylmaleimide/hydroxyethyl maleimide copolymer; and copolymers of vinyl pyrrolidone, vinyl acetate and vinyl propionate.

11. The stable foamed composition as defined in claim 1, wherein said at least one inert gas is selected from the group consisting of nitrogen, carbon dioxide and noble gases.

12. The stable foamed composition as defined in claim 1, substantially free of anionic surface-active compounds.

13. The stable foamed composition as defined in claim 1, having a foam degree of at least 10% after storage for at least one week at 20° C.

14. The stable foamed composition as defined in claim 1, made by a method comprising the steps of:
a) preparing an unfoamed composition comprising said water, said at least one alkoxylated silicone compound and optionally at least one consistency-imparting ingredient, wherein said at least one consistency imparting substance is at least one waxy substance that is solid at 25° C. and/or at least one thickener; and wherein said at least one alkoxylated silicone compound consists of at least one of said bis-alkoxylated silicone compounds, said alkoxylated silicone waxes, said water-insoluble alkoxylated silicone compounds and said esters of fatty acids and alkoxylated silicone compounds;
b) subsequently or simultaneously heating the unfoamed composition to a temperature above a melting point of said at least one waxy substance when said at least one waxy substance is present; and
c) subsequently or simultaneously foaming the unfoamed composition with said air and/or said at least one inert gas or forcing said air and/or said at least one inert gas into said unfoamed composition.

15. The stable foamed composition as defined in claim 1, further comprising at least one active ingredient selected from the group consisting of pharmaceutically effective ingredients, dermatologically effective ingredients, skin care effective ingredients, hair care effective ingredients, permanent wave effective ingredients, hair dye compounds and hair dye pre-cursors.

16. The stable foamed composition as defined in claim 15, wherein said at least one active ingredient is selected from the group consisting of plant extracts, vegetable extracts, protein hydrolyzates, silk hydrolyzates, light-protective agents, anti-oxidants, radical-trapping agents, anti-flaking agents, luster-imparting agents, vitamins, panthenol, softeners, combability-improving agents, skin moisturizing agents, proteins, insect repellants, bactericides, anti-viral agents, anti-microbial agents, proteolytically effective substances, keratolytically acting substances, keratin-reducing substances, oxidizing agents, direct-dyeing dye compounds and oxidation dye pre-cursor compounds.

17. The stable foamed composition as defined in claim 15, and in the form of a stable foamed cosmetic skin cream containing at least one skin care ingredient;
a stable foamed dermatological or medicinal salve containing at least one pharmaceutical or dermatological effective ingredient acting on skin or absorbed by the body through the skin when the salve is applied to the skin;
a stable foamed sunscreen cream containing at least one organic or inorganic UV filter;
a stable foamed hairstyling cream containing at least one hair-fixing substance;
a stable foamed hair care cream containing at least one hair care substance;
a stable foamed cream for permanent wave treatments containing at least one permanent wave effective ingredient;
a stable foamed cream for hair smoothing containing at least one hair smoothing ingredient;
or a stable foamed hair coloring cream containing at least one direct-dyeing hair dye compound or at least one oxidation dye precursor.

18. The stable foamed composition as defined in claim 1, in the form of a hair care composition, and further comprising at least one hair care effective ingredient, and wherein said at least one hair care effective ingredient is at least one cationic surfactant, at least one amine-substituted surfactant, at least one cationic silicone compound, at least one amine-substituted silicon compound, cationic polymers and/or at least one amine-substituted polymers.

19. A method of hair care, hairstyling, hair cleansing and/or body cleansing, said method comprising the steps of providing the stable foamed composition as defined in claim 1 in a package and using the stable foamed composition provided in the package for hair care, hairstyling, hair cleansing and/or body cleansing.

20. A method of using the stable foamed composition as defined in claim 1 in a suitable package to make a stable foamed cosmetic skin cream containing at least one skin care ingredient; a stable foamed dermatological or medicinal salve containing at least one pharmaceutical or dermatological effective ingredient acting on skin or absorbed by the body through the skin when the salve is applied to the skin; a stable foamed sunscreen cream containing at least one organic or inorganic UV filter; a stable foamed hairstyling cream containing at least one hair-fixing substance; a stable foamed hair care cream containing at least one hair care substance; a stable foamed cream for permanent wave treatments containing at least one permanent wave effective ingredient;
a stable foamed cream for hair smoothing containing at least one hair smoothing ingredient or a stable foamed hair coloring cream containing at least one direct-dyeing hair dye compound or at least one oxidation dye precursor.

21. A method of stabilizing a foamed composition, said method comprising including a foam stabilizer in said composition and wherein said foamed stabilizer is at least one alkoxylated silicone compound and said at least one alkoxylated silicone compound consists of at least one of said bis-alkoxylated silicone compounds, said alkoxylated silicone waxes, said water-insoluble alkoxylated silicone compounds and said esters of fatty acids and alkoxylated silicone compounds.

22. A method of making a stable foamed composition as defined in claim 1, said method comprising the steps of:
a) preparing an unfoamed composition comprising said water, said at least one alkoxylated silicone compound and optionally at least one consistency-imparting ingredient, wherein said at least one consistency imparting substance is at least one waxy substance that is solid at 25° C. and/or at least one thickener; and wherein said at least one alkoxylated silicone compound consists of at least one of said bis-alkoxylated silicone compounds, said alkoxylated silicone waxes, said water-insoluble alkoxylated silicone compounds and said esters of fatty acids and alkoxylated silicone compounds;
b) subsequently or simultaneously heating the unfoamed composition to a temperature above a melting point of said at least one waxy substance when said at least one waxy substance is present; and
c) subsequently or simultaneously foaming the unfoamed composition with said air and/or said at least one inert gas or forcing said air and/or said at least one inert gas into said unfoamed composition.

23. The method as defined in claim 22, wherein said forcing or foaming comprises rapidly stirring the unfoamed composition with a stirring device.

24. The method as defined in claim 22, wherein said foaming of the unfoamed composition is performed by a mixer and wherein said mixer comprises a mixing head and a feed device with respective connectors through which said unfoamed composition and said air and/or said at least one inert gas are correspondingly supplied.
